(19) United States
(12) Patent Application Publication
(10) Pub. No.: US 2010/0274098 A1
(43) Pub. Date: Oct. 28, 2010

(54) Patient Monitoring System

(75) Inventors: Nancy Belford, Fremont, CA (US); Todd Fjield, Laguna Hills, CA (US); Kathryn Willybiro, Huntington Beach, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/604,615

(22) Filed: Oct. 23, 2009

Related U.S. Application Data
(60) Provisional application No. 61/107,983, filed on Oct. 23, 2008.

(51) Int. Cl. A61B 5/00

(52) U.S. Cl. 600/300

(57) ABSTRACT

A patient monitoring system is disclosed having a central configuration management system in communication with a plurality of medical monitors via a communication system. The central communication management system allows users to create a plurality of use-specific monitor configurations, such as physician-specific and location-specific monitor configurations. The plurality of use-specific monitor configurations are stored on the central communication management system until a use-specific monitor configuration is selected for a particular monitor. The communication system is then used to transmit the selected use-specific monitor configuration from the central configuration management system to the particular monitor. A use-specific configuration can be selected for a monitor based on, for example, a selection made by a user of the monitor or automatically based on an RFID tag associated with a particular physician or location proximate to the monitor.

### CLAIM OF PRIORITY UNDER 35 U.S.C. §119

This application claims priority to U.S. Provisional Application No. 61/107,983, filed Oct. 23, 2008 entitled Patient Monitoring System and assigned to the assignee hereof and hereby incorporated by reference herein.

### FIELD

In general, embodiments of the invention relate to systems, methods, and computer program products for monitoring patients and, more particularly, relate to systems, methods, and computer program products for configuring patient monitors.

### BACKGROUND

Medical monitors, including patient monitors, are used in hospitals and other medical facilities to monitor and display one or more medical parameters, such as a patient's vital signs. Accordingly, medical monitors include or are configured to communicate with one or more sensors or other medical devices to receive information about one or more medical parameters. The medical monitors also typically include a display that shows the status of the one or more monitored parameters in real-time or near real-time. Some medical monitors are equipped with alarms to notify nearby medical personnel if a parameter being monitored exceeds or falls below a predefined threshold, as the case may be.

Some monitors have limits or settings that are user-configurable. For example, Vigilance™ Monitors offered by Edwards Lifesciences, which monitor and display the status of one or more patient parameters, allow a user to customize the display settings and limits. Traditionally, however, each monitor must be separately configured based on the function of the medical device or the preferences of the user of the medical device. If a medical device is used for a different function or by a different user, each setting and/or limit on the device may need to be individually adjusted as appropriate for the new function or user. Individually configuring and reconfiguring the many monitors and/or other medical devices in a hospital or other medical setting is labor intensive and creates a risk that costly errors may occur.

### SUMMARY

A patient monitoring system is disclosed having a central configuration management system in communication with a plurality of medical monitors via a communication system. The central communication management system allows users to create a plurality of use-specific monitor configurations, such as physician-specific and location-specific monitor configurations. The plurality of use-specific monitor configurations are stored on the central communication management system until a use-specific monitor configuration is selected for a particular monitor. The communication system is then used to transmit the selected use-specific monitor configuration from the central configuration management system to the particular monitor. A use-specific configuration can be selected for a monitor based on, for example, a selection made by a user of the monitor or automatically based on an RFID tag associated with a particular physician or location and located proximate to the monitor.

More particularly, embodiments of the present invention provide a patient monitoring system having a memory device and a processing device communicatively coupled to the memory device. The memory device has a plurality of predefined use-specific monitor configurations stored therein. The processing device is configured to receive information about how a monitor is to be used and then select a monitor configuration from the plurality of predefined use-specific monitor configurations based on the information received about how the monitor is to be used.

In one embodiment of the system, the predefined use-specific monitor configurations include user-specific monitor configurations, and the information about how the monitor is to be used includes information about a user that will use the monitor. For example, in one embodiment, the predefined use-specific monitor configurations include physician-specific monitor configurations, and the information about how the monitor is to be used comprises information about a particular physician that will use the monitor. In some embodiments, the predefined use-specific monitor configurations include location-specific monitor configurations, and the information about how the monitor is to be used includes information about a location in which the monitor will be used. In some embodiments, the predefined use-specific monitor configurations include patient-specific monitor configurations, and the information about how the monitor is to be used includes information about a patient with whom the monitor will be used. In some embodiments, the predefined use-specific monitor configurations include procedure-specific monitor configurations, and the information about how the monitor is to be used includes information about a procedure with which the monitor will be used. In some embodiments, the predefined use-specific monitor configurations comprise treatment protocol-specific monitor configurations, and the information about how the monitor is to be used comprises information about a treatment protocol with which the monitor will be used. In some embodiments, the predefined use-specific monitor configurations comprise medical device-specific monitor configurations, and the information about how the monitor is to be used comprises information about a medical device with which the monitor will be used.

In one embodiment, the patient monitoring system includes a configuration management application embodied as computer-readable instructions stored on a computer-readable medium. In such an embodiment, the processor and the configuration management application are configured such that the processor executing the computer-readable instructions is configured to receive user input having configuration information and to store the configuration information in the memory device as one of the plurality of predefined use-specific configurations. In some embodiments, the processor and the configuration management application are configured such that the processor executing the computer-readable instructions is configured to receive user input having configuration information and to compare the received configuration information with one or more configuration rules stored in the memory device.

In one embodiment, the patient monitoring system includes a patient monitor and the monitor includes the display device, the processing device, and the memory device described above.

In another embodiment, however, the patient monitoring system includes a plurality of monitors, a communication system such as a network, and a central configuration management device configured to communicate with the plurality of monitors via the communication system. In such an embodiment, the central configuration management device includes the memory device and the processing device described above, and the communication system is configured to transmit the selected monitor configuration from the central configuration management device to one of the plurality of monitors. Furthermore, in one embodiment, one or more of the plurality of monitors comprises an input device configured to receive information about how the one of the plurality of monitors is to be used, and the communication system is configured to transmit the information about how the one of the plurality of monitors is to be used from the one of the plurality of monitors to the central configuration management device.

In one embodiment, the patient monitoring system further includes an input device for receiving the information about how a monitor is to be used from a machine-readable code located proximate to the monitor. The machine-readable code located proximate to the monitor may include, for example, a radio frequency identification signal or a barcode associated with a patient, a medical professional, a medical device, a medical procedure, a treatment protocol, and/or a location proximate to the monitor.

In one embodiment, the plurality of predefined use-specific monitor configurations comprise monitor display settings or monitor alarm settings. For example, the configuration of a monitor may include specifications related to the such things as the alarm volume, alarm thresholds, alarm types, graph axes, limits, display colors, display layout, languages, data sources, headings, units, number formats, and the like.

Embodiments of the present invention further provide a system for managing a plurality of medical monitors, the system including a central processing device, a plurality of medical monitors, and a communication system for transferring information between the central processing device and the plurality of medical devices, where the central processing device is configured to track current configuration settings of each of the plurality of medical monitors. In one embodiment, the central processing system stores a plurality of predefined use-specific monitor configurations and is configured to use the communication system to communicate one or more of these predefined use-specific monitor configurations to a particular monitor based on use information received from an input device associated with the monitor. The predefined use-specific monitor configurations stored by the central processing system may include one or more configurations customized for a particular medical professional, medical procedure, monitor location, patient, medical device used in conjunction with the monitor, treatment protocol, or patient type. In some embodiments, the system includes a machine-readable code reader associated with at least one of the plurality of medical monitors and configured to receive use information from a machine-readable code provider, such as a RFID tag or barcode, placed proximate to the machine-readable code reader.

Embodiments of the invention also provide a medical monitor for monitoring one or more physical characteristics of a patient. In one embodiment, the medical monitor includes a display, an input interface, and a processor communicatively coupled to the display and the input interface. The medical monitor is configured to receive information from the input interface about use of the monitor and the processor is configured to automatically adjust one or more of its settings based on a predefined configuration selected based on the received information about the use of the monitor. In one embodiment of the monitor, the input interface includes a machine-readable code reader configured to receive information about use of the monitor from a machine-readable code provider, such as an RFID tag or barcode, placed proximate to the machine-readable code reader. In one embodiment, the machine-readable code provider is associated with a particular medical professional, and the predefined configuration selected based on the received information about the use of the monitor comprises a configuration customized for the particular medical professional.

Embodiments of the invention also provide a computer program product for managing the configuration settings of one or more medical monitors. The computer program product includes a computer-readable medium having computer-readable program code portions embodied therein. In one embodiment, the computer-readable program code portions include: (1) a first executable portion configured to allow for the creation of a plurality of use-specific configurations for a medical monitor, and (2) a second executable portion configured to distribute one of the plurality of use-specific configurations to a specific medical monitor based on a received indication of a use parameter related to a manner in which the specific medical monitor is to be used.

In one embodiment of the computer program product the use parameter comprises information specifying a medical professional that will be using the monitor. In other embodiments, the use parameter comprises information specifying a particular patient with whom the monitor will be used, a location in which the monitor will be used, a procedure with which the monitor will be used or a medical sensing device that will be used with the monitor.

In one embodiment of the computer program product, the first executable portion described above includes an executable portion configured to compare a use-specific configuration with one or more configuration rules to determine if the use-specific configuration is acceptable. In some embodiments, the first executable portion includes an executable portion that allows a user to select or specify a type of medical monitor, and an executable portion that provides the user with one or more customizable configuration settings based on the type of medical monitor specified by the user.

In some embodiments, the computer program product further includes an executable portion configured to track configuration changes in a plurality of monitors in a medical facility.

Embodiments of the invention also provide a method for customizing the settings of a medical monitor for a particular use. In one embodiment, the method includes: (1) creating a plurality of use-specific configurations for the medical monitor; (2) storing the plurality of use-specific configurations on a computer-readable storage device; and (3) distributing at least one of the plurality of use-specific configurations to the medical monitor based on a determination about a planned use for the medical monitor. The method may further include receiving use information from an input device associated with the monitor, and making the determination about the planned use for the medical monitor based on the received use information. The method may also include displaying on the monitor a list of pre-defined use-specific configurations, wherein receiving use information comprises receiving a user selection of a use-specific configuration displayed on the monitor. In one embodiment, the determination about the planned use for the medical monitor comprises an identification of a particular medical professional that will use the medical monitor.

### BRIEF DESCRIPTION OF THE DRAWINGS

Having thus described embodiments of the invention in general terms, reference will now be made to the accompanying drawings, which are not necessarily drawn to scale, and wherein:

FIG. 1 is a block diagram illustrating a medical monitor in accordance with an embodiment of the present invention;

FIG. 2 is a block diagram illustrating a patient monitoring system and monitor configuration management system in accordance with an embodiment of the present invention;

FIG. 3 is a flow chart illustrating the process of creating a customized use-specific configuration, in accordance with an embodiment of the present invention; and

FIG. 4 is a flow chart illustrating use of the configuration management system of FIG. 2 to reconfigure a monitor based on a use parameter, in accordance with an embodiment of the present invention.

### DETAILED DESCRIPTION OF EMBODIMENTS OF THE INVENTION

Embodiments of the present invention now will be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Embodiments of the present invention are directed to systems, methods, and computer program products for monitoring patients and for configuring patient monitors or other medical devices. In one embodiment of the invention, a plurality of medical monitors are communicatively coupled to a central monitor configuration management system, where the central management system stores various settings and other configuration information for the monitors and communicates the proper settings or other configuration information to each monitor.

Embodiments of the present invention further provide systems, methods, and computer program products for allowing a user to create and save one or more predefined use-specific monitor configurations that are customized based on at least one particular use parameter. For example, embodiments of the present invention store physician-specific, location-specific, patient-specific, medical device-specific, treatment protocol-specific, and/or procedure-specific monitor configurations and allow a user to select one of the predefined monitor configurations from the monitor or other location. In one embodiment, a predefined monitor configuration is automatically selected for a particular monitor based, for example, on the proximity of a radio frequency identification (RFID) tag to the monitor. Some embodiments of the present invention further include a computer program product that determines if the changes to one or more configuration settings comply with one or more configuration rules. Although embodiments of the invention are generally described herein as involving medical monitors, other embodiments of the present invention may be used to configure other medical devices.

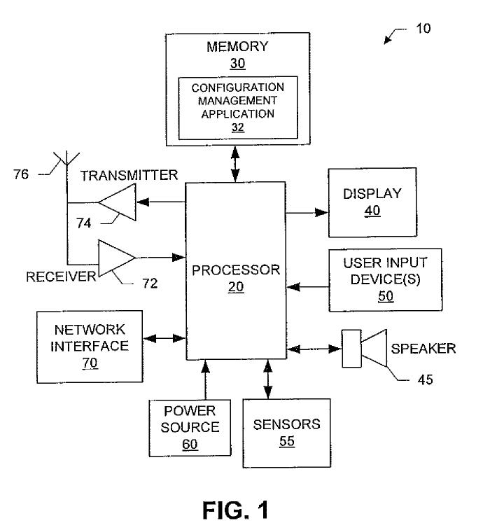

FIG. 1 illustrates a block diagram of a medical monitor 10, such as a patient monitor, in accordance with an embodiment of the present invention. Patient monitors are generally configured to monitor one or more physical characteristics of a patient. While several embodiments of a medical monitor 10 are described herein for purposes of example, other types of medical devices can readily employ embodiments of the present invention. Such devices may or may not be monitoring devices.

As illustrated in FIG. 1, the monitor 10 includes a processor 20. It is understood that the processor 20 includes circuitry required for implementing audio, visual, and logic functions of the monitor 10. For example, the processor 20 may be comprised of a digital signal processor device, a microprocessor device, and various analog-to-digital converters, digital-to-analog converters, and other support circuits. Control and signal processing functions of the monitor 10 are allocated between these devices according to their respective capabilities. The processor 20 may include functionality to operate one or more software programs based on computer-readable instructions thereof, which may be stored in memory 30. In one embodiment of the invention, the memory 30 includes a configuration management application 32 stored therein for configuring the monitor's settings and limits, as described in greater detail below. Some embodiments may include other computer programs. For example, the processor 20 may be capable of operating a connectivity program, such as a conventional Web browser. The connectivity program may then allow the monitor 10 to transmit and receive Web content, such as location-based content, according to a Wireless Application Protocol (WAP), for example.

In general, the memory 30 is communicatively coupled to the processor 20. The memory 30 may include volatile memory, such as volatile Random Access Memory (RAM) including a cache area for the temporary storage of data. The monitor 10 may also include other non-volatile memory, which can be embedded and/or may be removable. The non-volatile memory can additionally or alternatively comprise an EEPROM, flash memory, or the like. The memories can store any of a number of pieces of information and data used by the monitor 10 to implement the functions of the monitor 10. For example, the memories can include an identifier, such as a serial number, capable of uniquely identifying the monitor 10 and/or the type of monitor 10.

The monitor 10 includes a communication interface including, for example, a network interface 70 and a user interface, operatively coupled to the processor 20. The network interface 70 may include a modem, server, or other electronic device that communicatively couples the monitor 10 to another electronic device or a network of devices, such as a local areal network (LAN), a wide area network (WAN), and/or a Global Area Network (GAN), such as the Internet. The network interface 70 may be configured for wireless or wireline communication. The user interface comprises one or more user output devices, such as a display 40 and a speaker 45 or other audio device (e.g., a ringer, buzzer, or bell). The user interface further comprises one or more user input devices 50, such as one or more keys or dials, a touch pad, touch screen, mouse, microphone, and/or the like.

In one embodiment, the communication interface further comprises an antenna 76 communicatively coupled to the processor 20 via a transmitter 74 and/or a receiver 72. Such a communication system may be a radio frequency identification (RFID) reader and/or writer for reading and/or writing information to or from a RFID tag. In other embodiments, the communication interface comprises an infrared transmitter and/or reader or other wireless communication systems.

The monitor 10 further includes one or more sensors 55 and/or one or more mechanisms for communicatively coupling the monitor 10 to one or more sensors 55. These sensors 55 generate information about the one or more parameters monitored by the monitor 10, such as the physical characteristics of a patient. For example, the sensors 55 may include heart pulse sensors, echocardiogram transducers, blood glucose sensors, blood oxygen sensors, blood pressure sensors, other hemodynamic sensors, brain wave sensors, body temperature sensors, continuous cardiac output sensors, mixed venous oxygen sensors, flow sensors, pressure transducers, hemoglobin sensors, or any other type of sensor. As further illustrated in FIG. 1, the monitor 10 also comprises a power source 60, such as a battery, alternating current source, or other power source.

In general, certain aspects of the monitor 10 are customizable. For example, the monitor 10 is typically configured to emit an alarm using the speaker 45 or another audio device when a certain monitored parameter or combination of parameters goes beyond, i.e. falls higher than or lower than, some predefined threshold or violates some predefined rule. The thresholds and rules for when the monitor 10 should sound an alarm are, in some embodiments, configurable and the alarm type and/or volume may also be configurable. For example, a medical practitioner may desire that a monitor being used in an emergency room on an adult have an alarm that goes off based on different thresholds or rules and at a different volume than that of a monitor being used on a newborn in a neonatal intensive care unit (NICU).

Many display settings of the monitor 10 are also customizable. For example, a medical practitioner may be able to select which monitored parameters are displayed on the monitor's display 40 and the layout of the parameters and zones on the display 40. The medical practitioner may be able to select certain parameters to be shown in number form and select others to be shown in graphical form. The medical practitioner may be able to select the particular color used to represent different monitored parameters on the display 40 and may be able to customize the presentation of other information such as the units used to describe a monitored parameter, the limits on the axes of a graph, the length of a time history shown for a monitored parameter, and the like. The medical practitioner 40 may also be able to customize backend configuration settings such as which information to record, which input/output terminals are used for which sensor devices, where on the network to send warning notifications if a certain parameter violates a certain rule, etc.

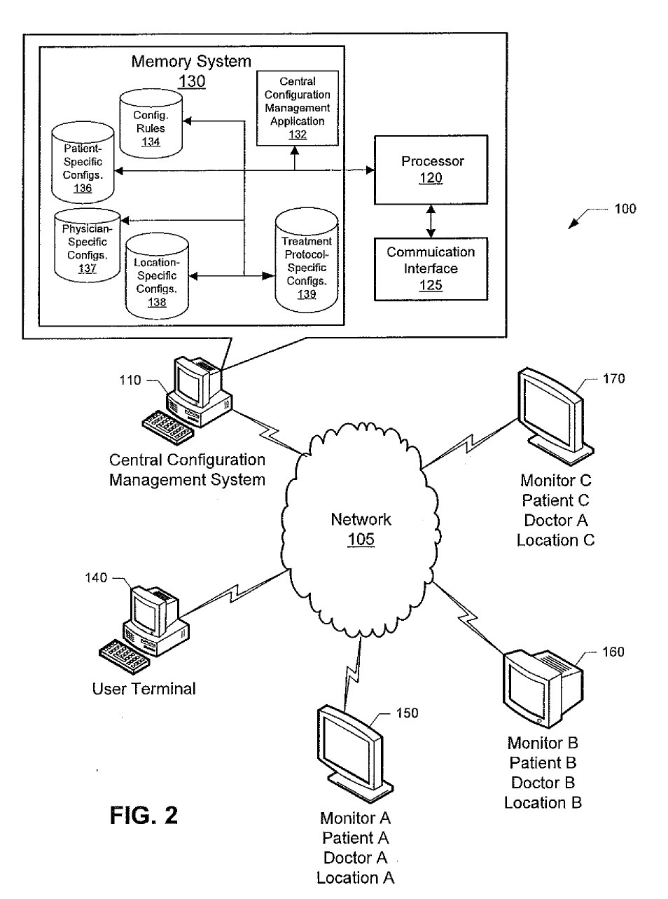

Referring now to FIG. 2, an illustration is provided of an exemplary system 100 for configuring and managing the configuration of medical monitors, in accordance with an embodiment of the invention. As illustrated, in one embodiment of the invention, the system 100 includes a plurality of medical monitors, such as “monitor A” 150, monitor “B” 160, and “monitor C” 170. Each medical monitor is generally structured as described above with reference to the monitor 10 of FIG. 1. In the illustrated embodiment of FIG. 2, monitor A 150 is monitoring patient “A” who is being treated by doctor “A” in location “A.” Monitor B 160 is monitoring patient “B” who is being treated by doctor “B” in location “B.” Monitor C 170 is monitoring patient “C” who is being treated by doctor “A” in location “C.” It should be appreciated that these pairings of monitors, doctors, patients, and locations are mere examples used below to aid in the description of the invention.

The system 100 further includes a central configuration management system 110 and one or more user terminals 140. In the illustrated embodiment, the three monitors 150, 160, and 170, and the user terminal 140, are communicatively coupled to the central configuration management system 110 via a network 105. As described above, the network 105 may include a WAN, LAN, or GAN, such as the Internet. The network 105 may be wired or wireless or involve a combination of wireline and wireless communications. In other embodiments, one or more of the monitors and/or one or more user terminals are communicatively coupled to the central configuration management system 110 via other communication systems. For example, in one embodiment of the invention, the communication system may even include a person carrying a portable computer-readable medium, such as a portable flash memory card, between the one or more monitors or terminals and the central configuration management system 110 to transport information from one device to the other.

The central configuration management system 110 generally comprises a processor 120, a communication interface 125, and a memory system 130. In one embodiment, the central configuration management system 110 comprises a personal computer, a server, or other computing device.

The processor 120 of the central configuration management system 110 is operatively coupled to the communication interface 125 and uses the communication interface 125 to communicate with the user terminal 140 and/or the monitors 150, 160, and 170. The communication interface 125 may include a modem, wireless antenna, wired connection device, or other systems for connecting to a network 105 or other communication system or electronic device.

The processor 120 of the central configuration management system 110 is also operatively coupled to the memory system 130. Like the memory 30 of the medical monitor 10 described above, the memory system 130 of the central configuration management system 110 may include volatile memory, such as volatile Random Access Memory (RAM) and/or non-volatile memory, which can be embedded and/or may be removable. The non-volatile memory can additionally or alternatively comprise an EEPROM, flash memory, or the like. The memory system 130 can store any of a number of pieces of information and data used by the central configuration management system 110 to implement the functions of the central configuration management system 110.

It should be noted that, although FIG. 1 illustrates a separate user terminal 140, monitor, and central configuration system, in some embodiments the user terminal 140 and the monitor are combined into a single device and/or the central configuration management system 110 is combined with the user terminal 140 and/or the monitor. For example, monitor “A” 150 could, in one embodiment, function as a monitor, user terminal 140, and central configuration system 110.

In the embodiment illustrated in FIG. 2, the memory system 130 includes a central configuration management application 132 embodied as computer-readable instructions stored in the computer-readable memory system 130. The processor 120 executes the computer-readable instructions of the central configuration management application 132 to perform certain procedures involved in managing the configuration of the monitors 150, 160, and 170. As will be described in more detail below, the memory system 130 further includes a configuration rules datastore 134 and one or more use-specific configuration datastores, such as a patient-specific configuration datastore 136, a physician-specific configuration datastore 137, a location-specific configuration datastore 138, and/or a treatment protocol-specific datastore 139. In other embodiments, other datastores may exist to store other predefined monitor configurations that are associated with other use parameters. For example, the memory system 130 may also include a medical device-specific datastore and/or a medical procedure-specific datastore.

In one embodiment of the system 100, the central configuration management system 110, monitor A 150, monitor B 160, monitor C 170, and the user terminal 140 are all located within the same hospital or other medical facility and connected to one another by the Internet or by the medical facility's own intranet. For example, in one embodiment, the medical monitors are located in different rooms or areas within the medical facility, while the central configuration management system 110 is located in some administrative or command and control location within the hospital. The user terminal 140 may be located in a computer laboratory of the medical facility or in the office of a physician or other medical professional. In other embodiments, however, one or more of the devices in the system 100 may not be located in the same medical facility and may be located more remotely from one another. For example, the user terminal 140 could be a physician's home computer and the central configuration management system 110 could be located remote from the medical facility where the monitors are located. In still other embodiments, the user terminal 140 and the monitor may be combined into a single device or the central configuration management system 110 may be combined with the user terminal 140 and/or the monitor, as described above.

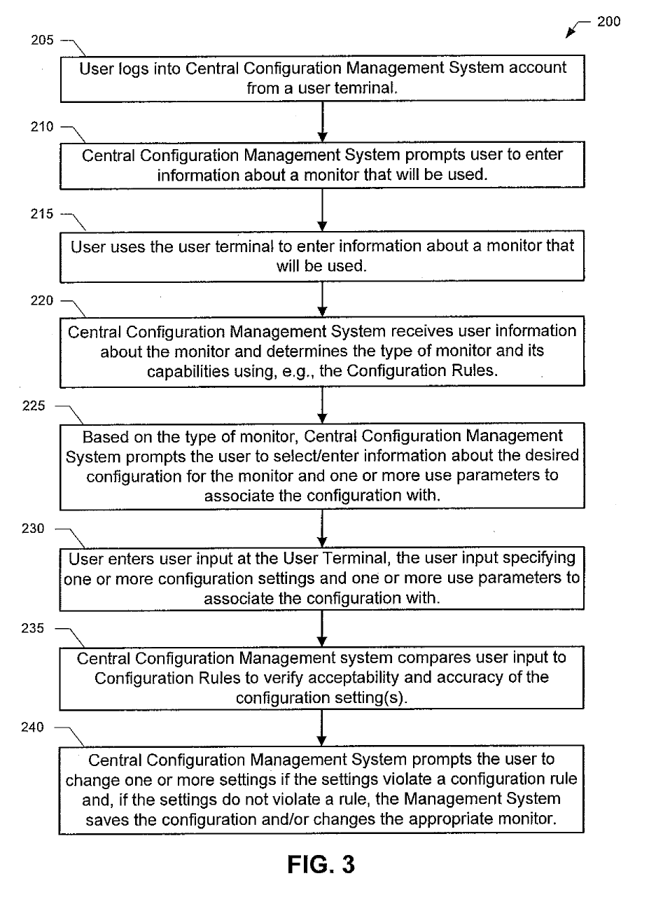

FIG. 3 illustrates a process 200 of creating one or more customized use-specific configurations. In the illustrated embodiment, a user accesses the central configuration management system 110 using the user terminal 140. In one embodiment, the user accesses the configuration management system 110 using a configuration management application stored in the memory of the user terminal 140. In other embodiments, the user utilizes a web browser on the user terminal 140 to access an Internet or intranet site hosted by the central configuration management system 110.

As illustrated by block 205 in FIG. 3, the central configuration management system 110 executes the central configuration management application 132 stored in its memory to prompt the user to log into an account on the central management system 110. In response, the user may use the user terminal 140 to enter a login ID and/or a password. For example, in one embodiment, all of the physicians and head nurses at a hospital are given the ability to access the central configuration management system 110. In some embodiments, the users each have their own separate accounts where they can save their own preferences and configurations. In other embodiments, however, the central configuration management system 110 is operated exclusively by one person, a small group of people, or a department in the medical facility and not by the individual practitioners.

If the user is able to log into or otherwise can access the central configuration management system 110, the central configuration management system 110 prompts the user to enter, and the user enters, information about a monitor that the user wants to update or about a monitor that will be used in the future, as illustrated by blocks 210 and 215. For example, in one embodiment, the central configuration management application 132 displays in a window on the display of the user terminal 140 a list of customizable monitors available in a particular facility. The user may then select a monitor from the displayed list of available monitors. In one embodiment, if a medical professional logs into his or her account, a list shows up that lists customizable monitors that exist within the facility in which the particular medical professional works.

In another embodiment, the central configuration management application 132 requests from the user, and the user enters, information about a particular monitor or the type of monitor. For example, the user may enter a monitor's serial number, manufacturer, name, model number, or some other identifier that can be used to identify the particular monitor and/or the type of monitor.

In still another embodiment, the user terminal 140 is combined with a monitor, such as monitor A 150. In other words, in one embodiment a user can access the central configuration management system 110 directly from a monitor, such as monitor A 150, to save a use-specific configuration for that monitor. In such an embodiment, the user may not have to enter any information about the monitor since, in one embodiment, the system 110 is configured to automatically recognize the monitor and/or type of monitor that is being used to access the system 110.

As illustrated by block 220, the central configuration management system 110 receives the user information about the monitor and, in one embodiment, uses this information to determine the type of medical monitor and its capabilities. For example, in one embodiment the user enters the serial number of a particular monitor, e.g., monitor A 150, and the central configuration management system 110 determines the type (e.g., the manufacturer and model) of monitor that monitor A 150 is and the capabilities of monitor A 150. In one embodiment, the central configuration management system 110 compares the inputted information about the monitor to a database of monitor types and capabilities. Such a database may be part of the configuration rules datastore 134 and/or may be maintained on some other remote system outside of, but communicatively coupled to, the central configuration management system 110. Knowing the type of monitor and, thus, its capabilities, the central configuration management system 110 can then determine which configuration settings exist for the monitor and which settings are customizable.

As represented by block 225, based on the type of monitor and its capabilities, the central configuration management system 110 prompts the user to select or otherwise enter information about the desired configuration for the monitor and one or more use parameters to associate the customized configuration with. For example, in one embodiment, the central configuration management system 110 displays for the user a list of the customizable configuration settings for the particular type of monitor and the current or default values for the monitor.

In one embodiment, the user specifies a use parameter to associate with the customized configuration. For example, the user may associate a new customized configuration with a particular person, a type of medical practitioner, a particular location in the medical facility, a particular medical procedure that the monitored patient is undergoing, a particular patient, a particular medical device, a treatment protocol, or a type of patient (i.e. a patient in a specified age range or with a certain condition, etc.). In other embodiments, however, the use parameter to be associated with a new customized configuration may be automatically determined. For example, where a user logs into his or her account on the central configuration management system 110 and generates a new customized configuration, the central configuration management system 110 may automatically associate the new configuration with that particular user or may set that user as the default use parameter.

As represented by block 230 of FIG. 2, after a user selects a customizable monitor setting, the user enters user input specifying the desired change to the setting. In some situations the user may specify the changes by making a selection from a list or dropdown menu of possible selections for each setting. For example, in one embodiment a monitor configuration tree, similar to a flowchart, is displayed to the user on the display of the user terminal. For each of the pathways in the tree, there may be several options for the user to choose from. These options may include conditionals that influence the configuration tree as the user approaches the end of each branch. The central configuration management application 132 may flag questionable entries or selections based on the configuration rules 134 and require confirmation from the user before acceptance.

For example, in one embodiment, the configuration management system 110 provides the user with options to create his/her own patient treatment protocol and then to distribute the treatment protocol to one or more monitors. In this regard, treatment protocol software executed on the configuration management system 110 may present the user with a “protocol tree” (similar to a flowchart) with conditionals the user selects that lead to a treatment option at the end of the tree.

In other situations, the user enters information into a text box displayed on the user terminal. In one embodiment, the user has the option of carrying the changes made to a particular monitor setting or group of settings through to other monitors with the same customizable configuration setting(s) as the monitor being changed.

As represented by block 235, the central configuration management system 110 compares the user input, i.e., the new or modified configuration setting(s), to one or more configuration rules stored in the configuration rule datastore 134 to determine if such a setting or change is acceptable and safe. For example, the configuration rules may check to see if a monitor can accept the desired setting or if the desired setting violates some law, standard, rule, or policy, such as a hospital safety policy that lists certain mandatory minimum monitor settings. For example, the configuration rules may require that a certain parameter always be displayed during a particular procedure or may not permit a user to deactivate certain alarms. The configuration rules may also check information such as the spelling of words entered as user input. In some embodiments, the central configuration management system 110 compares the user input to the configuration rules as the user is entering the input, while, in other embodiments, the system 110 compares the entire new monitor configuration with the rules 134 just before the new configuration is saved in a datastore in memory 130. In some embodiments, the configuration rule datastore 134 also includes rules related to acceptable programmable treatment protocols.

As represented by block 240, if one or more of the new configuration settings violates a configuration rule, the system 110 may either prompt the user to change the setting or, in some instances, automatically change the setting to the previous setting or to a default setting. If, however, the desired configuration settings are acceptable, the central configuration management system 110 saves the configuration to a datastore in the memory system 130. The central configuration management system 110 may also be configured to, at the request of the user, send the new configuration to a particular monitor to be changed and to track the changes to the configuration of this monitor and any other monitor. In this regard, one embodiment of the central configuration management control system 110 functions as a command center where the configuration settings for a plurality of medical monitors can be changed and/or tracked at a single location in real-time or near real-time.

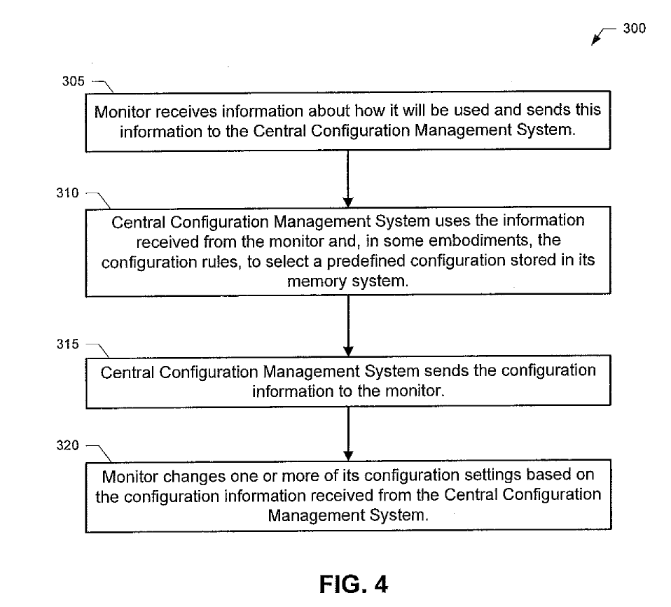

FIG. 4 provides a flow chart illustrating the process 300 of using the configuration management system of FIG. 2 to reconfigure a medical monitor based on a use parameter, in accordance with an embodiment of the present invention. More specifically, FIG. 4 illustrates an embodiment of the invention where a medical monitor can be reconfigured based on a predefined use-specific configuration, where the predefined use-specific configuration is chosen based on information received at the monitor.

As illustrated by block 305, the medical monitor, such as monitor A 150, receives information about how the monitor is to be used, referred to herein as “use information,” and sends this information to the central configuration management system 110. The use information may be information about the particular medical practitioner (e.g., a physician, nurse, etc.) that will use the monitor, a type of medical practitioner that will use the monitor (e.g., a specific type of doctor, surgeon, nurse, etc.), a particular location in the medical facility (e.g., the emergency room, NICU, burn center, operating room, bedroom, labor and delivery room, etc.), a particular medical procedure that the monitored patient is undergoing, a particular medical device that will be used in conjunction with the monitor, a treatment protocol, a particular patient, or a type of patient (i.e. a patient in a specified age range, with a certain condition, etc.).

In one embodiment, the monitor prompts the user on the monitor's display to enter use information using a user input device, such as a keyboard or touch screen. In such an embodiment, the user may simply type. For example, a medical practitioner who knows he has a predefined monitor configuration stored in the central configuration management system 110 may simply type his or her name into a field on the monitor's display.

In another embodiment, the monitor communicates with the central configuration management system 110 to display a list of use parameters that have predefined configurations associated therewith. For example, the monitor may prompt a user to search for a particular monitor configuration by first asking the user to select a certain type of use-specific configuration from a list of possible use-specific configuration types, such as for example, physician-specific configurations, location-specific configurations, and procedure-specific configurations. If the user selects “physician-specific configurations,” the monitor displays a list of physicians who have one or more customized monitor configurations associated with them. Selecting on a physician's name may select a particular configuration associated with that name or may display a list of configurations associated with that name. For example, selecting physician A from the list may cause the monitor to display a list of configurations associated with physician A, which may include, for example, a general configuration for type A monitors, a general configuration for type B monitors, a configuration for type A monitors during procedure X, a configuration for type A monitors during procedure Y, etc. A user can then select a configuration to use for the current monitor from this list of configurations. After a user enters or makes a selection of one or more use parameters or use-specific configurations, this “use information” is sent to the central configuration management system 310.

In other embodiments, the use information is entered into the monitor when a machine-readable code reader associated with the monitor reads a machine-readable code located proximate to the reader and/or monitor. For example, in one embodiment the machine-readable code comprises an RFID signal received from an RFID tag. For instance, in one embodiment, a surgeon carries a card having an RFID tag therein. When the surgeon is preparing for surgery, the surgeon can hold his card near a RF reader associated with the monitor and the central configuration management system 110 will identify him as the medical practitioner using the monitor and select the surgeon's predefined monitor configuration for the monitor. In other embodiments, the machine-readable code comprises a barcode, a two-dimensional bar code, an infrared code, an alphanumeric code, or some other machine-readable code.

In still other embodiments or instances, use information may be entered from a remote terminal 140 instead of from the monitor. For example, in one embodiment the surgeon described above can use the system 110 to configure and reconfigure the monitor(s) in the operating room from his or her office before going into the operating room. In such a situation, the user can configure a monitor by selecting a predefined monitor configuration stored in the memory system 130 of the central configuration management system 110 and associating it with the monitor, as described above.

A illustrated by block 310, the central configuration management system 110 uses the information received from the monitor and, in some embodiments, the configuration rules, to select a predefined configuration stored in its memory system 130. In embodiments of the invention where a user selects a configuration name from a list of possible predefined configurations shown on the display of the monitor, then the processor 120 of the central configuration management system 110 finds the configuration information associated with the selected configuration name. In embodiments where other use information is entered, such as the name of a physician or procedure or a code associated with some machine-readable code, the processor 120 searches the datastores in the memory 130 for configurations associated with the use information. In some embodiments, the processor 120 then compares the sleeted configuration information with one or more configuration rules 134 stored in the memory 134 to determine if the settings of the selected configuration are compatible with the particular monitor being configured or other rules, regulations, policies, etc.

As represented by block 315, the central configuration management system 110 then sends the configuration information to the monitor. This communication is accomplished through a communication system such as the network 105. As represented by block 320, once the medical monitor receives the configuration information from the central configuration management system 110, the monitor changes one or more of its settings to be consistent with the received configuration information,

In some embodiments, the central configuration management system 110 may receive use information associated with a single monitor where the use information includes information about more than one use parameter. In such an embodiment, the central configuration management system 110 may use the more than one use parameters to select a predefined monitor configuration.

For example, referring again to FIG. 2, in one embodiment, patient A has a RFID tag associated with him or her indicating the age of the patient, doctor A has a RFID tag associated with him or her uniquely identifying the doctor, and the room in which the monitor is located, i.e., location. A, also has a RFID tag associated with it. In such an embodiment, a RFID reader associated with or part of the monitor A 150 may read all three RFID tags and send the information to the central configuration management system 110 as use information. The central configuration management system 110 may also receive an identifier from the monitor 150 uniquely indentifying the monitor 150 or the type of monitor. With all of this information, the central management system 110 may select, for example, a predefined configuration specific to doctor A when doctor A is performing a procedure in location A and using a monitor of the same type as monitor A as opposed to a predefined configuration for doctor A when doctor A is, for example, performing a procedure in location C. In some embodiments, where there are conflicts in predefined settings, such as where there is not a predefined monitor configuration for the combination of doctor A and location A, but where there are separate predefined configurations for doctor A and location A, the configuration rules 134 are used to resolve conflicts between the two configurations.

As will be appreciated by one of skill in the art, the present invention may be embodied as a method, system (including an apparatus), computer program product, or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product comprising a computer-readable storage medium having computer-usable program code/computer-readable instructions embodied in the medium.

Any suitable computer-readable medium may be utilized. The computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CD-ROM), or other tangible optical or magnetic storage device; or transmission media such as those supporting the Internet or an intranet. Note that the computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

In the context of this document, a computer-readable medium may be any medium that can contain, store, communicate, propagate, or transport the program for use by or in connection with the instruction execution system, platform, apparatus, or device. The computer-readable medium may include a propagated data signal with the computer-readable program instructions embodied therewith, either in baseband or as part of a carrier wave. The computer-readable instructions may be transmitted using any appropriate medium, including but not limited to the Internet, wireline, optical fiber cable, radio frequency (RF), or the like.

Computer-readable instructions for carrying out operations of the present invention may be written in an object oriented, scripted or unscripted programming language such as Java, Peri, Smalltalk, C++, or the like. However, the computer-readable instructions for carrying out operations of the invention may also be written in conventional procedural programming languages, such as the “C” programming language or similar programming languages.

Embodiments of the present invention are described above with reference to flowchart illustrations and/or block diagrams of methods, apparatus (systems) and computer program products according to embodiments of the invention. It will be understood that each block of the flowchart illustrations and/or block diagrams shown in FIGS. 3 and 4, and combinations of blocks in the flowchart illustrations and/or block diagrams, can be implemented by computer-readable instructions. These computer-readable instructions may be provided to a processor of a general purpose computer, special purpose computer, or other programmable data processing apparatus to produce a machine, such that the instructions, which execute via the processor of the computer or other programmable data processing apparatus, create a mechanism for implementing the functions/acts specified in the flowchart and/or block diagram block or blocks.

These computer-readable program instructions may also be stored in a computer-readable memory that can direct a computer or other programmable data processing apparatus to function in a particular manner, such that the instructions stored in the computer-readable memory produce an article of manufacture including instruction mechanisms which implement the function/act specified in the flowchart and/or block diagram block or blocks.

The computer-readable program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the flowchart and/or block diagram block or blocks. Alternatively, computer program implemented steps or acts may be combined with operator or human implemented steps or acts in order to carry out an embodiment of the invention.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A patient monitoring system comprising:
a memory device having a plurality of predefined use-specific monitor configurations stored therein; and
a processing device communicatively coupled to the memory device, wherein the processing device is configured to receive information about how a monitor is to be used, and wherein the processing device is configured to select a monitor configuration from the plurality of predefined use-specific monitor configurations based on the information received about how the monitor is to be used.

2. The patient monitoring system of claim 1, wherein the predefined use-specific monitor configurations comprise user-specific monitor configurations, and wherein the information about how the monitor is to be used comprises information about a user that will use the monitor.

3. The patient monitoring system of claim 1, wherein the predefined use-specific monitor configurations comprise physician-specific monitor configurations, and wherein the information about how the monitor is to be used comprises information about a particular physician that will use the monitor.

4. The patient monitoring system of claim 1, wherein the predefined use-specific monitor configurations comprise location-specific monitor configurations, and wherein the information about how the monitor is to be used comprises information about a location in which the monitor will be used.

5. The patient monitoring system of claim 1, wherein the predefined use-specific monitor configurations comprise patient-specific monitor configurations, and wherein the information about how the monitor is to be used comprises information about a patient with whom the monitor will be used.

6. The patient monitoring system of claim 1, wherein the predefined use-specific monitor configurations comprise procedure-specific monitor configurations, and wherein the information about how the monitor is to be used comprises information about a procedure with which the monitor will be used.

7. The patient monitoring system of claim 1, wherein the predefined use-specific monitor configurations comprise treatment protocol-specific monitor configurations, and wherein the information about how the monitor is to be used comprises information about a treatment protocol with which the monitor will be used.

8. The patient monitoring system of claim 1, wherein the predefined use-specific monitor configurations comprise medical device-specific monitor configurations, and wherein the information about how the monitor is to be used comprises information about a medical device with which the monitor will be used.

9. The patient monitoring system of claim 1, further comprising:
a monitor, wherein the monitor comprises a display device, the processing device, and the memory device.

10. The patient monitoring system of claim 1, further comprising:
a configuration management application embodied as computer-readable instructions stored on a computer-readable medium, wherein the processor and the configuration management application are configured such that the processor executing the computer-readable instructions is configured to receive user input having configuration information and store the configuration information in the memory device as one of the plurality of predefined use-specific configurations.

11. The patient monitoring system of claim 1, further comprising:
a configuration management application embodied as computer-readable instructions stored on a computer-readable medium, wherein the processor and the configuration management application are configured such that the processor executing the computer-readable instructions is configured to receive user input having configuration information and compare the received configuration information with one or more configuration rules stored in the memory device.

12. The patient monitoring system of claim 1, further comprising:
a plurality of monitors;
a communication system; and
a central configuration management device configured to communicate with the plurality of monitors via the communication system, wherein the central configuration management device comprises the memory device and the processing device, and wherein the communication system is configured to transmit the selected monitor configuration from the central configuration management device to one of the plurality of monitors.

13. The patient monitoring system of claim 12, wherein the one of the plurality of monitors comprises an input device configured to receive information about how the one of the plurality of monitors is to be used, and wherein the communication system is configured to transmit the information about how the one of the plurality of monitors is to be used from the one of the plurality of monitors to the central configuration management device or to another of the plurality of monitors.

14. The patient monitoring system of claim 12, wherein the communication system comprises a network.

15. The patient monitoring system of claim 1, further comprising an input device for receiving the information about how a monitor is to be used from a machine-readable code located proximate to the monitor.

16. The patient monitoring system of claim 15, wherein the machine-readable code located proximate to the monitor comprises a radio frequency identification signal associated with a patient, a medical professional, a medical procedure, a medical device, a treatment protocol, or a location proximate to the monitor.

17. The patient monitoring system of claim 1, wherein the plurality of predefined use-specific monitor configurations comprise monitor display settings, monitor alarm settings, monitor help settings, or monitor treatment guidance settings.

18. A system for managing a plurality of medical monitors, the system comprising:
a central processing device;
a plurality of medical monitors; and
a communication system for transferring information between the central processing device and the plurality of medical devices,
wherein the central processing device is configured to track current configuration settings of each of the plurality of medical monitors.

19. The system of claim 18, wherein the central processing system stores a plurality of predefined use-specific monitor configurations and is configured to use the communication system to communicate one or more of these predefined use-specific monitor configurations to a particular monitor based on use information received from an input device associated with the monitor.

20. The system of claim 19, wherein the predefined use-specific monitor configurations stored by the central processing system comprises one or more configurations customized for a particular medical professional, medical procedure, monitor location, patient, patient type, medical device used in conjunction with the monitor, or treatment protocol.

21. The system of claim 19, further comprising:
a machine-readable code reader associated with at least one of the plurality of medical monitors and configured to receive use information from a machine-readable code provider placed proximate to the machine-readable code reader.

22. The system of claim 19, wherein the configuration settings comprise at least one of an alarm volume, alarm threshold, alarm type, graph parameter, display color, display layout, language, data source, unit of measure, or monitor display screen.

23. A medical monitor for monitoring one or more physical characteristics of a patient, the medical monitor comprising:
a display;
an input interface; and
a processor communicatively coupled to the display and the input interface, wherein the medical monitor is configured to receive information from the input interface about use of the monitor, and wherein the processor is configured to automatically adjust one or more of its settings based on a predefined configuration selected based on the received information about the use of the monitor.

24. The medical monitor of claim 23, wherein the input interface comprises:
a machine-readable code reader configured to receive information about use of the monitor from a machine-readable code provider placed proximate to the machine-readable code reader.

25. The medical monitor of claim 24, wherein the machine-readable code provider is associated with a particular medical professional and wherein the predefined configuration selected based on the received information about the use of the monitor comprises a configuration customized for the particular medical professional.

26. The medical monitor of claim 24, wherein the machine-readable code provider is associated with a particular medical procedure, monitor location, patient, patient type, medical device used in conjunction with the monitor, or treatment protocol, and wherein the predefined configuration selected based on the received information about the use of the monitor comprises a configuration customized for the particular medical procedure, monitor location, patient, patient type, medical device used in conjunction with the monitor, or treatment protocol.

27. A computer program product for managing the configuration settings of one or more medical monitors, the computer program product comprising computer-readable medium having computer-readable program code portions embodied therein, the computer-readable program code portions comprising:
a first executable portion configured to allow for the creation of a plurality of use-specific configurations for a medical monitor;
a second executable portion configured to distribute one of the plurality of use-specific configurations to a specific medical monitor based on a received indication of a use parameter related to a manner in which the specific medical monitor is to be used.

28. The computer program product of claim 27, wherein the use parameter comprises information specifying a medical professional that will be using the monitor.

29. The computer program product of claim 27, wherein the use parameter comprises information specifying a particular patient with whom the monitor will be used.

30. The computer program product of claim 27, wherein the use parameter comprises information specifying a location in which the monitor will be used.

31. The computer program product of claim 27, wherein the use parameter comprises information specifying a procedure with which the monitor will be used.

32. The computer program product of claim 27, wherein the use parameter comprises information specifying one or more medical device(s) that will be used with the monitor.

33. The computer program product of claim 27, wherein the use parameter comprises information specifying one or more treatment protocol(s) that will be used with the monitor.

34. The computer program product of claim 27, wherein the first executable portion configured to allow for the creation of a plurality of use-specific configurations for a medical monitor further comprises:
an executable portion configured to compare a use-specific configuration with one or more configuration rules to determine if the use-specific configuration is acceptable.

35. The computer program product of claim 27, further comprising:
an executable portion configured to track configuration changes in a plurality of monitors in a medical facility.

36. The computer program product of claim 27, wherein the first executable portion configured to allow for the creation of a plurality of use-specific configurations for a medical monitor further comprises:
an executable portion that allows a user to select or specify a type of medical monitor; and
an executable portion that provides the user with one or more customizable configuration settings based on the type of medical monitor specified by the user.

37. A method for customizing the settings of a medical monitor for a particular use, the method comprising:
creating a plurality of use-specific configurations for the medical monitor;
storing the plurality of use-specific configurations on a computer-readable storage device; and
distributing at least one of the plurality of use-specific configurations to the medical monitor based on a determination about a planned use for the medical monitor.

38. The method of claim 37, further comprising:
receiving use information from an input device associated with the monitor; and
making the determination about the planned use for the medical monitor based on the received use information.

39. The method of claim 38 further comprising:
displaying on the monitor a list of pre-defined use-specific configurations, wherein receiving use information comprises receiving a user selection of a use-specific configuration displayed on the monitor.

40. The method of claim 37, wherein the determination about the planned use for the medial monitor comprises an identification of a particular medical professional that will use the medical monitor, a medical procedure, a monitor location, a patient, a patient type, a medical device used in conjunction with the monitor, or a treatment protocol.
